(19) United States
(12) Patent Application Publication
(10) Pub. No.: US 2013/0059362 A1
(43) Pub. Date: Mar. 7, 2013

(54) BIOSENSORS COMPRISING PROTEIN-BINDING DOMAINS AND FLUORESCENT PROTEINS

(71) Applicants: John Fetter, St. Louis, MO (US); Dmitry Malkov, St. Louis, MO (US); Nathan Zenser, St. Louis, MO (US); Keming Song, St. Louis, MO (US)

(72) Inventors: John Fetter, St. Louis, MO (US); Dmitry Malkov, St. Louis, MO (US); Nathan Zenser, St. Louis, MO (US); Keming Song, St. Louis, MO (US)

(73) Assignee: SIGMA-ALDRICH CO., LLC, St. Louis, MO (US)

(21) Appl. No.: 13/641,050

(22) PCT Filed: Apr. 13, 2011
(86) PCT No.: PCT/US2011/032214

§ 371 Date: Nov. 19, 2012

Related U.S. Application Data
(60) Provisional application No. 61/323,698, filed on Apr. 13, 2010.
(60) Provisional application No. 61/323,719, filed on Apr. 13, 2010.
(60) Provisional application No. 61/323,702, filed on Apr. 13, 2010.
(60) Provisional application No. 61/367,017, filed on Jul. 23, 2010.
(60) Provisional application No. 61/390,668, filed on Oct. 7, 2010.
(60) Provisional application No. 61/408,856, filed on Nov. 1, 2010.
(60) Provisional application No. 61/431,957, filed on Jan. 12, 2011.

(51) Int. Cl. C12N 5/071; C12N 7/01

(52) U.S. Cl. 435/235.1; 435/325; 435/371

(57) ABSTRACT

The present invention provides compositions and methods for monitoring cellular signal transduction events. In particular, signal transduction events may be monitored by the use of biosensors comprising protein-binding domains, which bind signal-transduction proteins, and fluorescent proteins. Provided herein, therefore, methods for detecting activation of signal transduction proteins or screening for agents that modulate the activity of signal transduction proteins. Also provided are cells comprising the biosensors; lentiviral particles comprising biosensor coding sequence; and kits comprising the lentiviral particles.

### CROSS-REFERENCE TO RELATED APPLICATIONS

This application claims the priority of U.S. provisional application No. 61/323,702, filed Apr. 13, 2010, U.S. provisional application No. 61/323,719, filed Apr. 13, 2010, U.S. provisional application No. 61/323,698, filed Apr. 13, 2010, U.S. provisional application No. 61/367,017, filed Jul. 23, 2010, U.S. provisional application No. 61/390,668, filed Oct. 7, 2010, U.S. provisional application No. 61/408,856, filed Nov. 1, 2010, and U.S. provisional application No. 61/431,957, filed Jan. 12, 2011, each of which is hereby incorporated by reference in its entirety.

### FIELD OF THE INVENTION

The present invention generally provides compositions and methods for monitoring cellular signal transduction events. More particularly, the present invention provides biosensors comprising protein-binding domains and fluorescent proteins and methods of using the biosensors for detecting changes in the activity of signal transduction proteins.

### BACKGROUND OF THE INVENTION

Activation of protein tyrosine kinases (PTKs) plays an important role in modulating a wide variety of cellular events, including differentiation, proliferation, migration, metabolism and apoptosis. Within the human genome, 90 PTK genes have been identified. Among all PTKs identified, 58 are receptor tyrosine kinases (RTKs). Activation of RTKs initiates a series of cellular responses to growth factors and hormones that reside in the cellular microenvironment, and therefore, are an important class of drug targets. For example, Epidermal Growth Factor Receptor (EGFR) is a RTK which has been implicated in human breast cancer development, and thus EGFR is one of the most studied RTKs.

RTKs are activated by the binding of specific ligands, which leads to dimerization of the receptors and autophosphorylation of specific tyrosine residues. These phosphotyrosine residues deliver signals to downstream molecules via their interactions with proteins containing Src homology 2 (SH2) domains. The protein-binding domain SH2 is approximately 100 amino acids long and recognizes phosphotyrosine residues within specific sequence contexts. SH2 domains are found in many intracellular proteins that generally function as adaptor proteins. For example, Grb2 (Growth factor binding protein 2) is a modular protein containing one SH2 domain that is constitutively associated with downstream regulators. Upon activation of EGFR, the SH2 domain of Grb2 binds a specific phosphorylated tyrosine residue in EGFR, thereby activating downstream kinases and intracellular events. More than 100 SH2 domains have been identified in proteins encoded by the human genome. The binding specificity of these domains plays a critical role in signal transduction within the cell, mediating the interaction of receptor proteins and other intermediary signaling molecules in response to changes in tyrosine phosphorylation states.

Several methods have been used to detect RTK activation or auto-phosphorylation upon ligand treatment. One of the most commonly used methods is antibody-based immunostaining that uses paired RTK-specific antibodies, one for detection of the total amount of RTK and the other for detection of phospho-RTK. Although widely used, the immunostaining-based method requires cell fixation and thus is not suitable for live cell imaging. The other commonly used method involves over-expression of an RTK-GFP fusion protein and tracks the distribution pattern of the fusion protein in live cells. This method, although useful in some cases, has two major drawbacks: 1) over-expression of an artificial target protein may compete against the endogenous protein and may also interfere with the physiological condition of the cell; and 2) tagging a fluorescent protein (>30 KD) to the target RTK protein could inactivate or alter the function of the RTK.

There is a need, therefore, for improved methods to monitor changes in RTK activities in real time in live cells with minimum disturbance to the cells. In addition, there is a need for rapid, high-throughput screening methods to identity pharmaceutical agents that affect RTK activities.

### SUMMARY OF THE INVENTION

Briefly, therefore, one aspect of the present disclosure provides a cell comprising a biosensor that is able to detect a change in activity of a signal transduction protein. The biosensor comprises at least one protein-binding domain and a fluorescent protein. Moreover, the cell expresses the biosensor at a level that is substantially similar to the level of expression of the signal transduction protein.

Another aspect of the disclosure encompasses a method for detecting activation of a signal transduction protein in real time. The method comprises providing a cell comprising a biosensor, wherein the biosensor comprises at least one protein-binding domain and a first fluorescent protein, and the protein-binding domain of the biosensor is able to bind to the signal transduction protein when the signal transduction protein is either activated or inactivated. Additionally, the cell expresses a biosensor at a level that is substantially similar to the level of the signal transduction protein. The method further comprises contacting the cell with a signal that activates the signal transduction protein such that binding between the protein-binding domain of the biosensor and the signal transduction protein changes. Lastly, the method comprises monitoring the biosensor in the cell, wherein a change in the location of the biosensor indicates activation of the signal transduction protein.

Still another aspect of the disclosure provides a method for determining whether an agent modulates the activity of a signal transduction protein in real time. The method comprises providing a cell comprising a biosensor, wherein the biosensor comprises at least one protein-binding domain and a first fluorescent protein, and the protein-binding domain of the biosensor is able to bind to the signal transduction protein when the signal transduction protein is either activated or inactivated. Additionally, the cell expresses a biosensor at a level that is substantially similar to the level of the signal transduction protein. The method further comprises contacting the cell with the agents. The next step of the method comprises contacting the cell with a signal that activates the signal transduction protein such that binding between the protein-binding domain of the biosensor and the signal transduction protein changes. The final step of the method comprises monitoring the biosensor in the cell relative to a control cell, wherein a change in the location of the biosensor in the cell contacted with the agent relative to the control cell indicates that the agent modulates the activity of the signal transduction protein.

A further aspect of the present disclosure provides a lentiviral particle comprising a nucleic acid encoding a biosensor. The biosensor comprises at least one protein-binding domain and a fluorescent protein.

Yet another aspect encompasses a kit for generating a cell comprising a biosensor. The kit comprises a plurality of cells and a plurality of lentiviral particles. Each lentiviral particle comprises a nucleic acid encoding the biosensor, which comprises at least one protein-binding domain and a fluorescent protein.

Other aspects and iterations of the disclosure are described in more detail below.

### REFERENCE TO COLOR FIGURES

The application file contains at least one photograph executed in color. Copies of this patent application publication with color photographs will be provided by the Office upon request and payment of the necessary fee.

### DESCRIPTION OF THE FIGURES

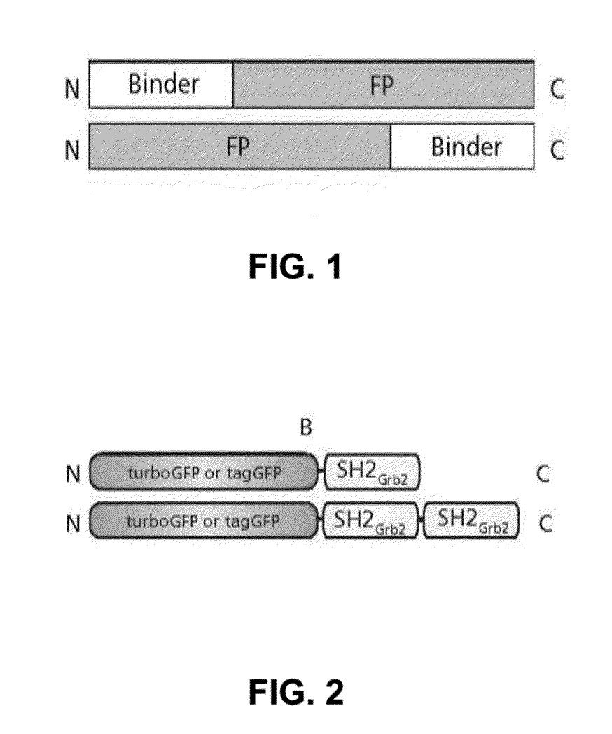

FIG. 1 illustrates the generic structure of biosensors that comprise a target-specific binding domain (i.e., “binder”) and a fluorescent protein (i.e., “FP”).

FIG. 2 presents the structure of two EGFR biosensors. Each biosensor comprises one or two SH2_{Grb2} domains and a green fluorescent protein tag (TurboGFP or TagGFP).

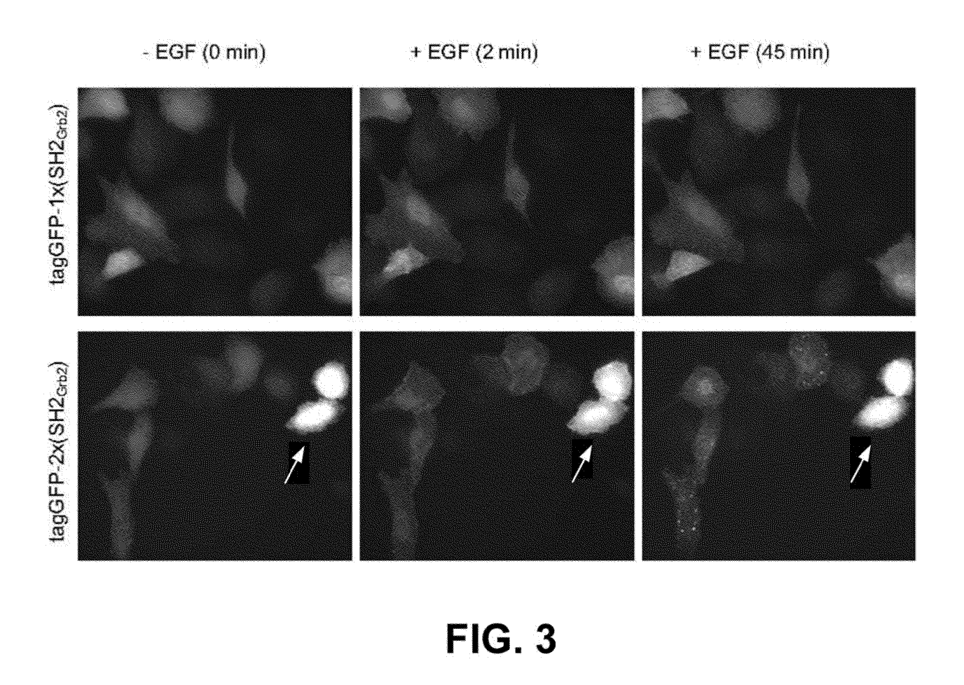

FIG. 3 illustrates that a biosensor comprising two SH2_{Grb2} domains has improved performance. Presented are fluorescence microscopy images of cells comprising a tagGFP-1×SH2Grb2 biosensor (top panels) and cells comprising a tagGFP-2×SH2_{Grb2} biosensor (bottom panels) in the absence or presence of 100 ng/mL of EGF.

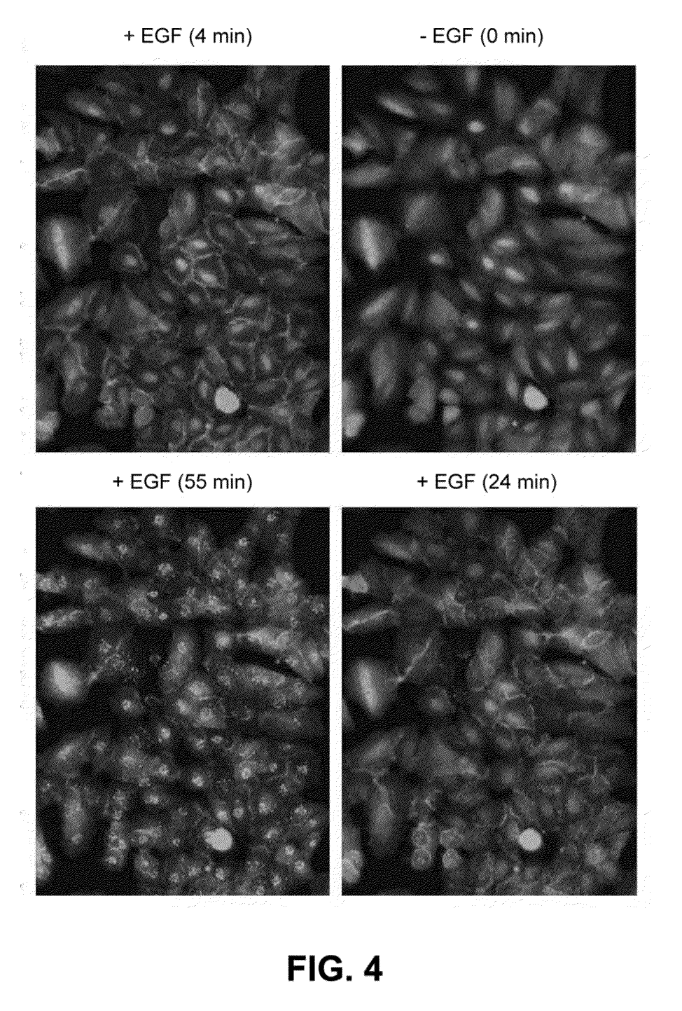

FIG. 4 depicts development of a stable cell line expressing a tagGFP-2×SH2_{Grb2} biosensor. Single cell clones were selected and assayed for biosensor activity with EGF.

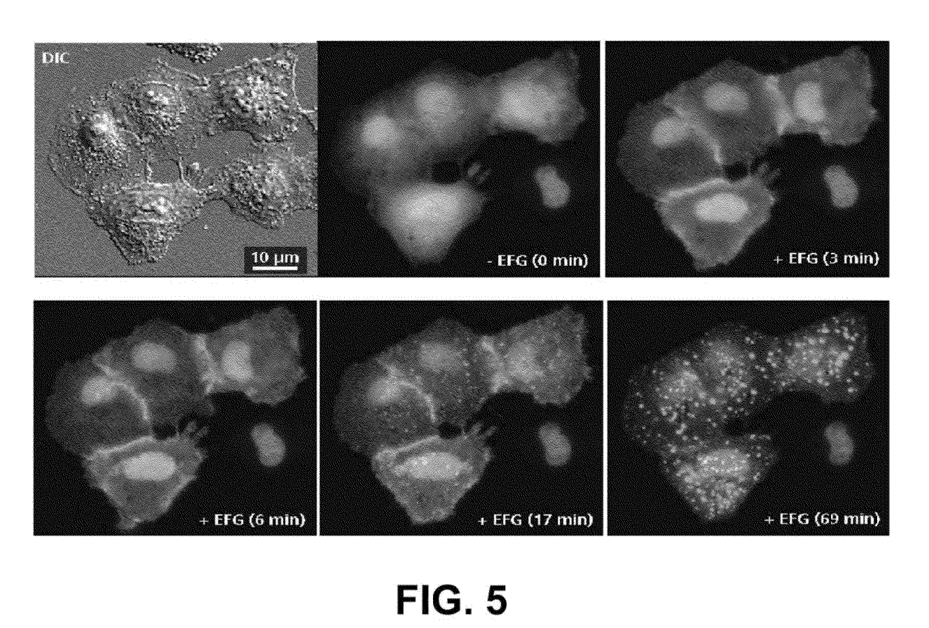

FIG. 5 depicts the TagGFP:2×SH2_{Grb2} biosensor translocation to the plasma membrane followed by internalization (granule formation) after treatment with 100 ng/mL EGF.

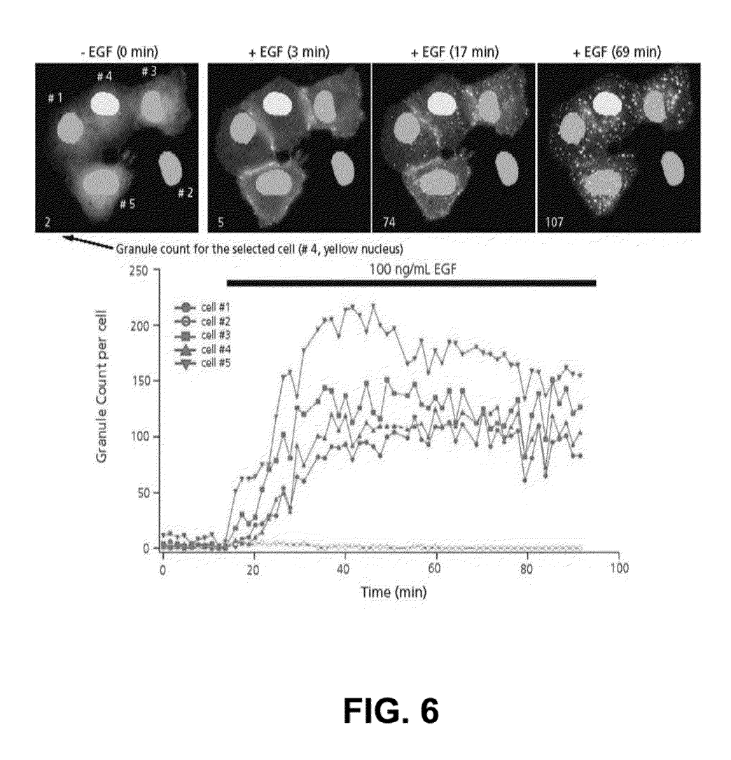

FIG. 6 documents the increasing biosensor internalization over time in individual cells. Individual cells are numbered in the left-most image in the panel of images. The number of granules per cell as a function of time is plotted at the bottom.

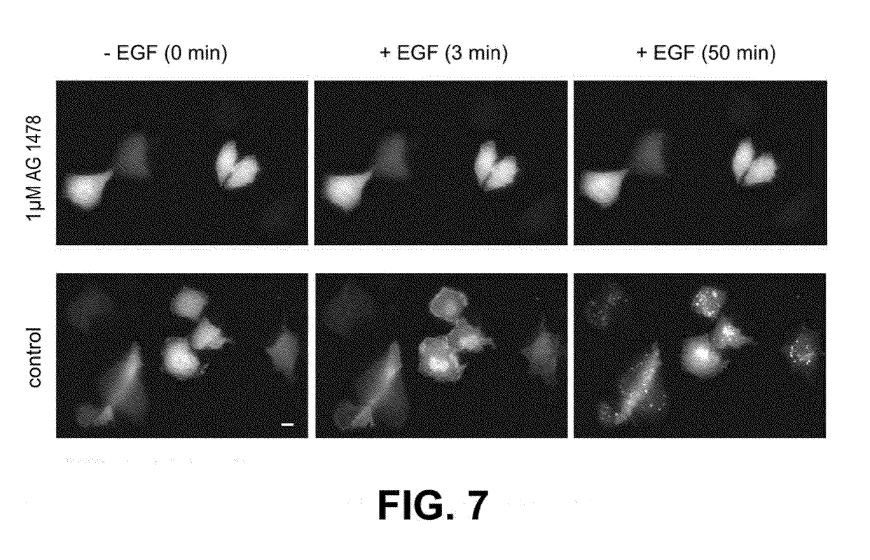

FIG. 7 demonstrates that tyrphostin AG 1478 inhibits relocalization of the TagGFP:2×SH2_{Grb2} biosensor. Presented are fluorescence microscopy images of cells treated with AG 1478 (upper panels) or control untreated cells (lower panels) in the absence or presence of 1 μg/mL of EGF. The bar represents 10 Ξm.

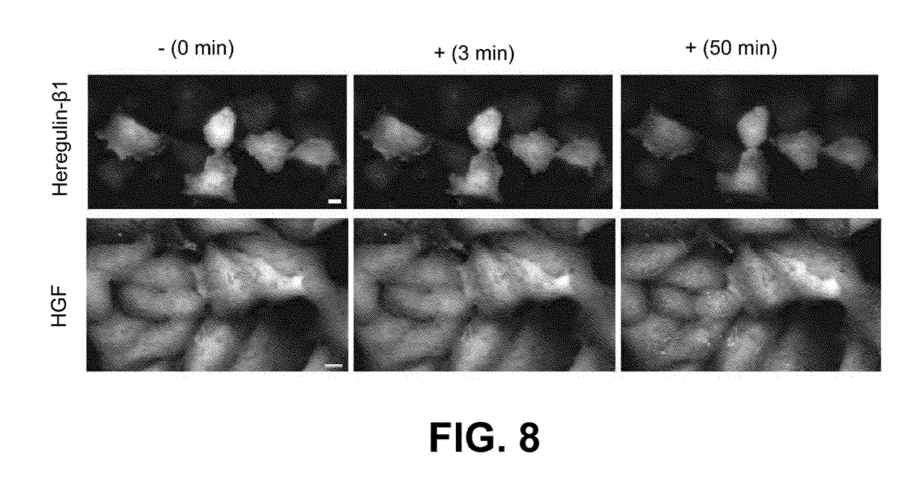

FIG. 8 illustrates the specificity of EGFR signaling pathway as monitored by relocalization of the 2×SH2_{Grb2} biosensors. Top panels show cells treated with 1 μg/mL of heregulin-61. Bottom panels show cells exposed to 100 ng/mL of HGF.

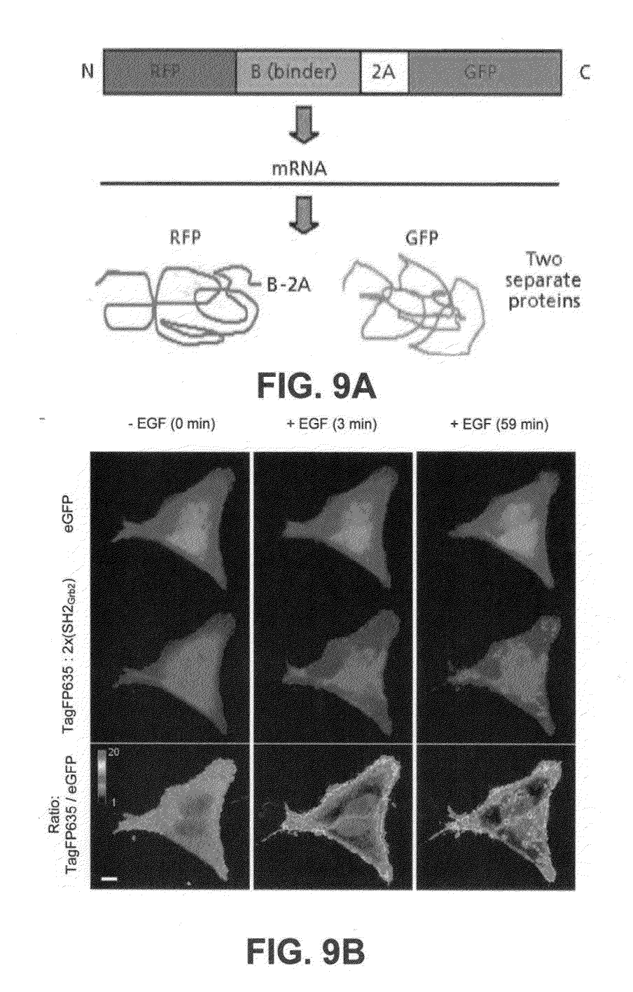

FIG. 9 demonstrates ratiometric imaging. (A) presents the structure of a construct in which a biosensor coding sequence is fused with a GFP sequence through the 2A sequence, and depicts the two separate proteins that are made during translation. (B) presents fluorescent images showing the location of each protein and the fluorescence ratio between the two in the absence and presence of 100 ng/mL of EGF. The bar represents 10 μm.

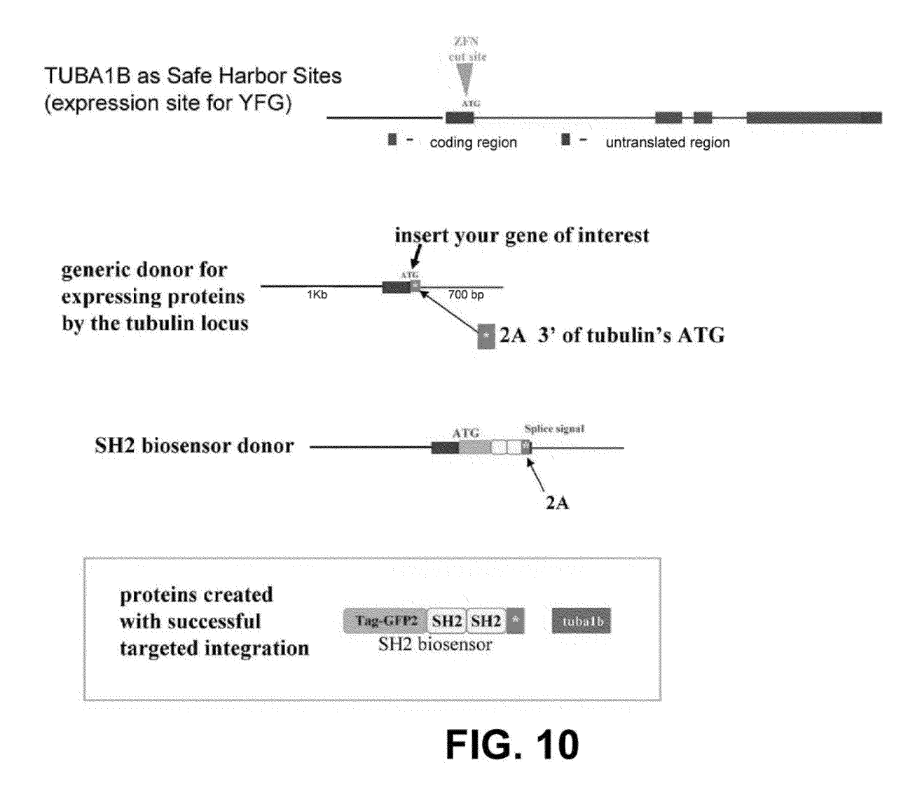

FIG. 10 depicts integration of a SH2 biosensor into the TUBA1B locus. Presented are schematics of the TUBA1B locus, site of integration, design of the SH2 biosensor, and the proteins expressed after successful integration.

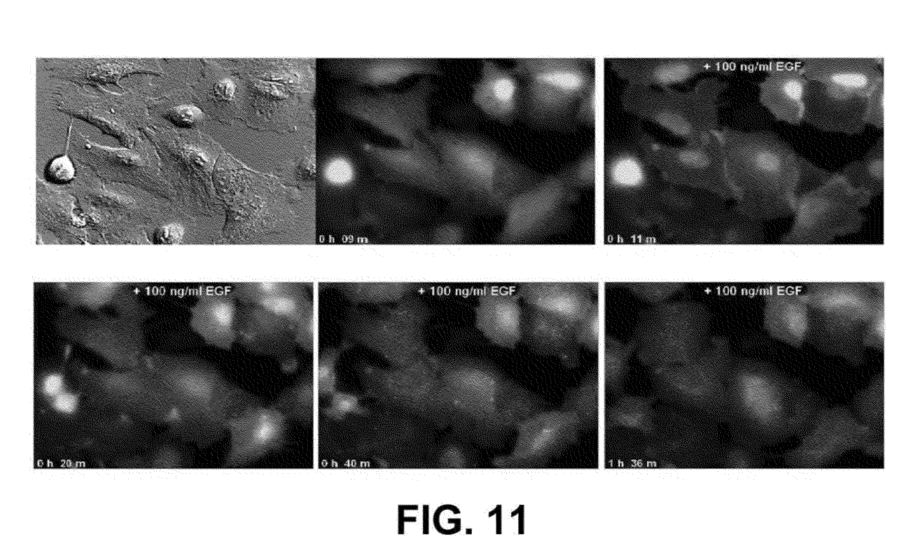

FIG. 11 presents differential interference contrast (DIC) and fluorescence microscopy images of individual isolated cell clones expressing the SH2 biosensor. Fluorescent images show a time course of SH2 biosensor translocation after exposure to 100 ng/mL of EGF.

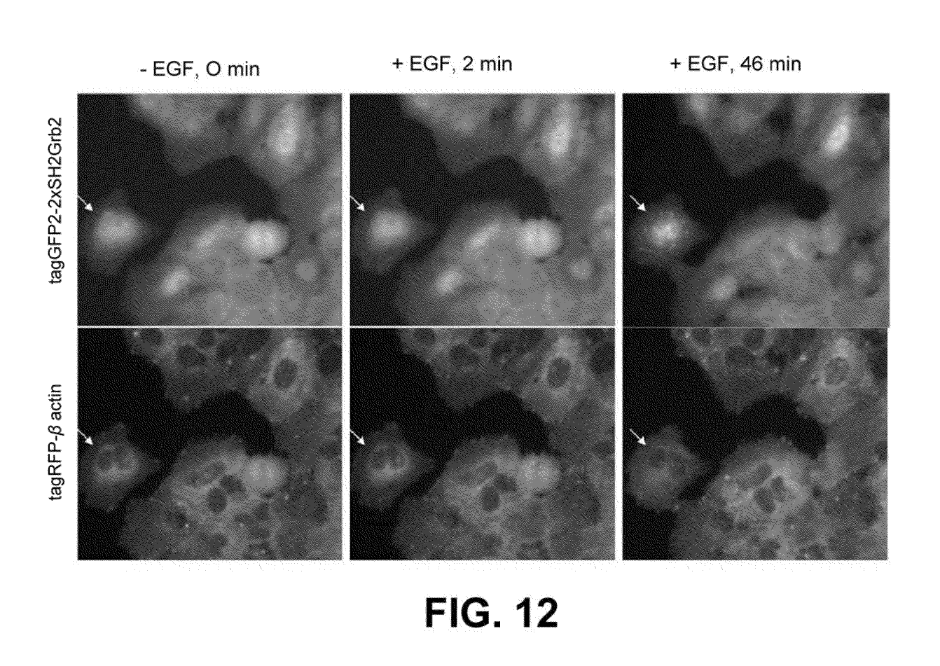

FIG. 12 depicts fluorescence microscopy images of individual isolated cell clones expressing the SH2 biosensor (upper panels) and RFP-actin (lower panels). Presented is a time course after exposure to 100 ng/mL of EGF.

### DETAILED DESCRIPTION OF THE INVENTION

The present disclosure provides biosensors comprising protein-binding domains and a fluorescent protein, wherein the biosensors are able to detect or “sense” changes in the activity of specific signal transduction proteins. In particular, the protein-binding domain(s) of the biosensor is able to bind a signal transduction protein that is either activated or inactivated, such that upon activation of the signal transduction protein, the cellular location of the biosensor changes. Additionally, the fluorescent protein of the biosensor provides means to track the change in location of the biosensor in response to activation of the signal transduction protein. Also provided herein are cells engineered to express the biosensor. Advantageously, it has been discovered that relocalization of the biosensor may be visualized in living cells if the cells express the biosensor at a level that is substantially similar to the level of the target signal transduction protein. The disclosure also provides methods for using the biosensor to detect activation of signal transduction proteins or screen for agents that modulate the activity of signal transduction proteins.

### (I) Biosensors

One aspect of the present disclosure encompasses a biosensor comprising at least one protein-binding domain and a fluorescent protein. Stated another way, the biosensor is a fusion protein comprising at least one protein-binding domain and a fluorescent protein. The protein-binding domain of the biosensor binds to a target protein (e.g., a signal transduction protein) under different conditions of activation, and the fluorescent protein of the biosensor provides means to detect and monitor the changes in the localization of the biosensor, which reflects changes in activity of the target protein. The biosensor, therefore, detects and monitors changes in the activity of the target signal transduction protein.

(a) Protein-Binding Domain

In general, a protein-binding domain is a structurally conserved region of a protein that recognizes and binds a specific site, a specific amino acid sequence, or a specific three-dimensional configuration in another protein. In the context of the present invention, a protein-binding domain is a structurally conserved region of a protein that recognizes and binds a specific site, a specific amino acid sequence, or a specific three dimensional configuration in a signal transduction protein. Protein-binding domains mediate many protein-protein interactions.

Numerous protein-binding domains and families of protein-binding domains and their target proteins have been identified and investigated. Many relational databases designed to store protein interactions mediated by protein-binding domains (or protein recognition modules) in human, mouse, rat, yeast, plant, and other organisms are available. For example, interactions mediated by as many as 150 different domain families are stored in DOMINO (http://mint.bio.uniroma2.1t/domino/); the largest database of human protein-protein interactions mediated by phosphoprotein binding domains (PPBDs) is the PepCyber: P˜Pep, which currently includes 11,269 records of interactions between 387 PPBD proteins and 1,471 substrate proteins, curated from 4,852 published studies (http://pepcyber.biolead.org/PPEP/).

Preferred protein-binding domains include SH3, SH2, 14-3-3, PDZ, PTB, WW, EVH, VHS, FHA, EH, FF, BRCT, Bromo, Chromo, GYF, C2, MH2, PBD, WD40, and variants thereof. In one embodiment, the preferred protein-binding domain may be a 14-3-3 domain, which binds to phosphoserine-containing proteins. In another embodiment, the protein-binding domain may be BRCT (BRCA1 C Terminus domain), which binds other BRCT modules to form homo/hetero BRCT multimers. In yet another embodiment, the protein-binding domain may be Bromo, a domain found in many chromatin-associated proteins and in nuclear histone acetyltransferases. Bromo interacts specifically with an acetylated lysine of a protein. In an additional embodiment, the protein-binding domain may be a C2 domain, which mostly are Ca2+-dependent membrane-targeting modules that bind phospholipids, inositol polyphosphates, and intracellular proteins. In another embodiment, the protein-binding domain may be Chromo (Chromatin Organization Modifier domain), which often binds to methylated histone tails. Another embodiment provides EH (Eps15 homology domain) as the protein-binding domain that recognizes proteins containing Asn-Pro-Phe (NPF) sequences. In one iteration, the protein-binding domain may be EVH (enabled/VASP homology) domain, which binds G protein-coupled receptor proteins. In another iteration, the protein-binding domain may be FF domain, which binds the hyperphosphorylated C-terminal repeat domain of RNA polymerase II. In an alternate embodiment, the protein-binding domain may be Forkhead associated domain, FHA, which binds phosphothreonine, phosphoserine and sometimes phosphotyrosine. In still another embodiment, the protein-binding domain may be GYF that contains conserved Gly-Tyr-Phe residues and is a proline-binding domain. In another embodiment, the protein-binding domain may be MAD homology 2 domain, MH2, which binds TGF-beta receptor kinase. In yet another embodiment, the protein-binding domain may be PBD (P21-Rho-binding domain), also known as CRIB (Cdc42/Rac interactive binding domain) that binds small GTPases. Alternatively, the protein-binding domain may be DHR (Dig homologous region) or GLGF (named after a conserved sequence motif). Through target phosphorylation, many PDZ domains bind C-terminal polypeptides and even to lipids, and PDZ-PDZ domain interactions lead to heterodimerization. In another embodiment, the protein-binding domain may PTB (Phosphotyrosine-binding domain), which binds phosphotyrosine with specificity conferred by residues located amino-terminal to the phosphotyrosine, as compared to SH2 domain's specificity conferred by the adjacent carboxy-terminal residue of phosphotyrosine. In other embodiments, the protein-binding domain may be SH3 or VHS. SH3 domains bind to peptides that are rich in proline, mostly containing the motif PxxP. VHS domains are involved in general membrane targeting/cargo recognition in vesicular trafficking.

In another embodiment, the protein-binding domain may be WD40. The 40 residues of a WD40 domain form a propeller-like structure to which proteins can bind either stably or reversibly, and the protein-protein interactions carry out functions in signal transduction, pre-mRNA processing and cytoskeleton assembly. In still another embodiment, the protein-binding domain may be a WW domain, which is a protein module with two highly conserved tryptophans that binds proline-rich peptide motif.

In a preferred embodiment, the protein-binding domain may be SH2, the Src homology 2 domain. SH2 is a phosphotyrosine binding domain of about 100 amino-acid residues first identified as a region with conserved sequence between the oncoproteins Src and Fps. Similar sequences have been found in many other intracellular signal-transducing proteins. SH2 domains are found in a wide variety of proteins, including phospholipase Cgamma (PLCG1) and the cellular non-receptor protein tyrosine kinases; structural proteins such as tensin (TNS1); a group of small adaptor molecules, i.e CRK and NCK1. Sometimes the SH2 domains are found as repeats in a single protein sequence and will then often bind both mono- and di-phosphorylated substrates. SH2 domains function as regulatory modules in intracellular signaling cascades and some other pathways, such as kinases, adaptors, phosphatases, and so on. In one embodiment, the SH2 domain may be from a natural protein or peptide. More specifically, in one preferred embodiment, the protein binding domain may be the SH2 domain contained in Grb2 (Growth factor binding protein 2). Grb2 is a modular protein that specifically binds to a phosphotyrosine residue in activated EGFR (Epidermal Growth Factor Receptor), via its SH2 domain, and is constitutively associated with other downstream regulators.

In some embodiments, the protein-binding domain may be from a natural protein. In other embodiments, the protein-binding domain may be a variant of a natural protein-binding domain, which includes but is not limited to truncated versions, mutated versions, modified versions, and a version with at least one conservative amino acids substitution. Conservative amino acid substitutions, as used herein, include amino acid residues that may be substituted with another amino acid residue having a similar side chain without affecting the function of the protein-binding domain. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acid substitution groups include: valine-leucine-isoleucine, phenylalanine-tyrosine, lysine-arginine, alanine-valine, and asparagine-glutamine.

In other embodiments, the protein-binding domain may be an artificial protein-binding domain that was designed to bind a specific region of the target protein of interest. In yet another embodiment, the protein-binding domain may be a hybrid of two or more protein-binding domains.

In general, the protein-binding domain or variant thereof may be derived from a human protein, a mouse protein, a rat protein, a mammalian protein, a vertebrate protein, an invertebrate protein, a microbial protein, a bacterial protein, or a viral protein. In an exemplary embodiment, the protein-binding domain or variant thereof may be from a human protein.

The number of protein-binding domains in a biosensor can and will vary. In one embodiment, the biosensor may comprise one protein-binding domain. In other embodiment, the biosensor may comprise two protein-binding domains. In a further embodiment, the biosensor may comprise three or more protein-binding domains. In embodiments in which the biosensor comprises more than one protein-binding domain, the protein-binding domains may be identical, the protein-binding domains may be from the same family, or the protein-binding domains may be from different families.

(b) Fluorescent Protein

The biosensor also comprises a fluorescent protein. Those of skill in the art appreciate that a variety of fluorescent proteins are suitable for inclusion in the biosensor.

Non limiting examples of suitable fluorescent proteins include green fluorescent proteins (e.g., GFP, GFP-2, tagGFP, turboGFP, EGFP, Emerald, Azami Green, Monomeric Azami Green, CopGFP, AceGFP, ZsGreen1), yellow fluorescent proteins (e.g. YFP, EYFP, Citrine, Venus, YPet, PhiYFP, ZsYellow1,), blue fluorescent proteins (e.g. EBFP, EBFP2, Azurite, mKalama1, GFPuv, Sapphire, T-sapphire,), cyan fluorescent proteins (e.g. ECFP, Cerulean, CyPet, AmCyan1, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein.

Non-limiting examples also include circular permutations of green fluorescent proteins, in which the amino and carboxyl portions are interchanged and rejoined with a short spacer connecting the original termini, while still being fluorescent. These circular permutations of fluorescent protein have altered pKa values and orientations of the chromophore. Furthermore, certain locations within some fluorescent protein tolerate insertion of entire proteins, and conformational changes in the insert can have profound effects on the fluorescence, such as enhancement or changed colors. For example, insertions of calmodulin or a zinc finger domain in place of Tyr-145 of a yellow mutant (EYFP, enhanced yellow fluorescent protein) of GFP result in indicator proteins whose fluorescence can be enhanced several fold upon metal binding. The calmodulin graft into enhanced yellow fluorescent protein can monitor cytosolic Ca²⁺ in single mammalian cells. In one possible embodiment, a protein-binding domain of a biosensor is inserted in fluorescent protein coding sequence.

(c) Biosensor Configurations

The orientation of the protein-binding domain(s) to the fluorescent protein of the biosensor can and will vary. As diagrammed in FIG. 1, the fluorescent protein may be located at the C-terminal end of the biosensor, or the fluorescent protein may be located at the N-terminal end of the biosensor.

In embodiments in which the biosensor comprises two or more protein-binding domains, the protein-binding domains may be arranged in tandem at either the N-terminal end or the C-terminal end of the biosensor. Alternatively, the two more protein-binding domains may flank the fluorescent protein. Stated another way, the fluorescent protein may be internal with protein-binding domains at the N-terminal and C-terminal ends of the biosensor.

(d) Exemplary Biosensors

In a preferred embodiment, the at least one protein-binding domain of the biosensor may be a SH2 domain. In another preferred embodiment, the biosensor may comprise two SH2 domains. In an iteration of this embodiment, the SH2 domains may be from a Grb2 protein. In an exemplary embodiment, the biosensor may comprise two SH2 domains derived from human Grb2. Exemplary fluorescent proteins include turboGFP and tagGFP. In one exemplary embodiment, the biosensor comprises two SH2 domains derived from human Grb2 linked to turboGFP or tagGFP, with the fluorescent protein at the N-terminal end of the biosensor.

### (II) Nucleic Acids and Vectors Comprising Nucleic Acids Encoding the Biosensor

(a) Nucleic Acids

Another aspect of the disclosure encompasses nucleic acids encoding the biosensors, which are detailed above in section (I). The nucleic acids encoding the biosensors may be deoxyribonucleic acids or ribonucleic acids. In some embodiments, the nucleic acids encoding the biosensors may be linear nucleic acid molecules. In other embodiments, the nucleic acids encoding the biosensors may be part of a vector, as detailed below.

(i) Expression Control Sequences

In some embodiments, the nucleic acid encoding the biosensor may be operably linked to an expression control sequence. Although expression is regulated by many sequence elements, the term “promoter” is used below for ease of discussion. In some embodiments it may be desirable to use a constitutive promoter that is active in many types of cells or under different conditions. Non-limiting examples of suitable constitutive promoters include CMV, PGK, SV40, MMTV, adenovirus Ela, immediate early, immunoglobulin heavy chain, and RSV-LTR promoters. In other embodiments, it may be desirable to use an inducible promoter such that expression of the biosensor may be easily regulated. Examples of suitable inducible promoters include tetracycline-inducible promoters and those regulated by metal ions (e.g., metallothionein-1 promoters), steroid hormones, small molecules, heat shock, and the like.

Those of skill in the art will appreciate that the nucleic acid encoding the biosensor may also be operably linked to other transcriptional and translational control elements. Suitable transcription or translation control sequences include but are not limited to upstream control elements, enhancer elements, TATA boxes, cis regulatory regions, activator binding regions, repressor binding regions, transcription initiation sites, polyadenylation control elements, transcription termination sites, ribosome binding sites, translation initiation sites, and translation termination sites.

(ii) Optional Sequence Encoding a Second Fluorescent Protein

In certain embodiments, the nucleic acid encoding the biosensor may be linked to a nucleic acid encoding a second fluorescent protein that differs from the fluorescent protein in the biosensor. Suitable fluorescent proteins are detailed above in section (I)(b). The nucleic acid encoding the biosensor and the nucleic acid encoding the second fluorescent proteins may be linked by a sequence encoding a 2A peptide, such that two separate proteins are made during translation. Alternatively, the nucleic acid encoding the biosensor and the nucleic acid encoding the second fluorescent proteins may be separated by an internal ribosome binding or entry site. Regardless of how the nucleic acid is constructed, the end result is that two separate proteins are made during translation (i.e., the biosensor and the second fluorescent protein), wherein the second fluorescent protein may be used as an internal standard or may be used for ratio imaging as detailed below.

As mentioned above, the nucleic acid encoding the biosensor and the nucleic acid encoding the second fluorescent proteins may be linked by a 2A coding sequence. As used herein, the term “2A peptide” refers to any 2A peptide or fragment thereof, any 2A-like peptide or fragment thereof, or an artificial peptide comprising the requisite amino acids. The 2A peptide was originally characterized in positive-strand RNA viruses, which produce a polyprotein that is “cleaved” during translation into mature individual proteins. More specifically, the 2A peptide region (˜20 amino acids) mediates “cleavage” at its own C-terminus to release itself from the 2B region of the polyprotein. 2A peptide sequences terminate with a glycine and a proline residue. During translation of a 2A peptide, the ribosome pauses after the glycine residue, resulting in release of the nascent polypeptide chain. Translation resumes, with the proline residue of the 2A sequence becoming the first amino acid of the downstream protein.

In some embodiments, the 2A peptide coding sequence that links the biosensor coding sequence with the second fluorescent protein coding sequence may code for a full length 2A peptide. In other embodiments, the 2A peptide coding sequence may code for a C-terminal fragment of a 2A peptide. The C-terminal fragment may comprise about 19, 18, 17, 16, 15, 14, 13, 12, 11, 10, 9, 8, 7, 6, 5, or 4 amino acid residues of the C-terminal end of the 2A peptide.

The 2A peptide coding sequence may be linked to the 5′ end or 3′ end of the nucleic acid encoding the biosensor. Accordingly, the resultant nucleic acid may have the following configurations: 5′-(biosensor sequence)-(2A peptide sequence)-(2^{nd} fluorescent protein sequence)-3′, or 5′-(2^{nd} fluorescent protein sequence)-(2A peptide sequence)-(biosensor sequence)-3′.

(b) Vectors

A further aspect of the disclosure provides vectors comprising the nucleic acid encoding the biosensor as detailed above in section (I). In some embodiments, the vector may comprise the nucleic acid encoding the biosensor. In other embodiments, the vector may comprise the nucleic acid encoding the biosensor that is operably linked to expression control sequences. In further embodiments, the vector may comprise the nucleic acid encoding the biosensor, which is operably linked to expression control sequences, as well as a nucleic acid encoding a second fluorescent protein.

The vector may be used to introduce the nucleic acid encoding the biosensor into a cell of interest and/or regulate expression of the biosensor in the cell of interest. Suitable vectors include plasmids, phagemids, cosmids, BACS, and viral vectors, such as, e.g., adenoviruses, adeno-associated viruses, herpes viruses, lentiviruses, retroviruses, and so forth.

In some embodiments, the vector comprising the nucleic acid encoding the biosensor and any optional accessory sequences may be an integrating vector. Integrating vectors not only introduce the nucleic acid of interest into a cell, but also integrate the nucleic acid of interest into a location in a chromosome of the cell. Typically, the nucleic acid of interest is integrated randomly into a site in the chromosome. Viral vectors are examples of integrating vectors.

In other embodiments, the vector comprising the nucleic acid encoding the biosensor and any optional accessory sequences may be an expression vector. Expression vectors generally contain origins of replication such that they remain extrachromosomal and regulate expression of the nucleic acid of interest. Expression vectors suitable for expression in eukaryotic cells including yeast, avian, and mammalian cells are known in the art. One example of an expression vector is pcDNA3 (Invitrogen, San Diego, Calif.), in which transcription is driven by the cytomegalovirus (CMV) early promoter/enhancer.

In a preferred embodiment, the vector comprising the nucleic acid encoding the biosensor and optional accessory sequences may be a lentiviral vector. An advantage of lentiviral vectors is that they are able to replicate in non-dividing cells. In an exemplary embodiment, the nucleic acid encoding the biosensor and any optional accessory sequences may be within a lentiviral particle. Lentiviral particles are ready to deliver and integrate the nucleic acid(s) of interest to a cell of interest. In some embodiments, the lentiviral particles may be highly purified. Methods of making lentiviral particles are well known in the art.

### (III) Kits for Generating Cells Comprising a Biosensor

A further aspect of the disclosure provides kits for generating cells comprising the disclosed biosensors. The kits comprise a plurality of vectors comprising the nucleic acid encoding the biosensor (and optional accessory sequences) and a plurality of cells to be transfected.

(a) Vectors

Suitable vectors comprising the nucleic acid encoding the biosensor and any optional accessory sequences are detailed above in section (II)(b). In an exemplary embodiment, the kits comprise lentiviral particles comprising the nucleic acid encoding the biosensor and any optional accessory sequences.

(b) Cells

A variety of cells are suitable for inclusion in the kits. Typically the cells provided in the kits will be cultured cells. Suitable cultured cells include human cell lines, mammalian cell lines, and non-mammalian cell lines. The cell line may be adherent or non-adherent, or the cell line may be grown under conditions that encourage adherent, non-adherent or organotypic growth using standard techniques known to individuals skilled in the art. Non-limiting examples of suitable mammalian cell lines include Chinese hamster ovary (CHO) cells, monkey kidney CVI line transformed by SV40 (COS-7), human embryonic kidney line 293, baby hamster kidney cells (BHK), mouse sertoli cells (TM4), monkey kidney cells (CVI-76), African green monkey kidney cells (VERO), human cervical carcinoma cells (HeLa), canine kidney cells (MDCK), buffalo rat liver cells (BRL 3A), human lung cells (W138), human liver cells (Hep G2), mouse mammary tumor cells (MMT), rat hepatoma cells (HTC), HIH/3T3 cells, the human U2-OS osteosarcoma cell line, the human A549 cell line, the human A-431 cell line, the human K562 cell line, the human HEK293 cell lines, the human HEK293T cell line, and TRI cells. For an extensive list of mammalian cell lines, those of ordinary skill in the art may refer to the American Type Culture Collection catalog (ATCC®, Mamassas, Va.). In an exemplary embodiment, the cells may be A549, A-431, HeLa or COS-7.

(c) Additional Components

In other embodiments, the kits may comprise targeting endonucleases or nucleic acids encoding targeting endonucleases such that the sequence encoding the biosensor may be targeted to a specific chromosomal sequence. The targeting endonuclease may be a naturally-occurring protein or an engineered protein. In some embodiments, the targeting endonuclease may be a meganuclease or a homing endonuclease. In other embodiments, the targeting endonuclease may be a transcription activator-like effector (TALE)-nuclease. In preferred embodiments, the targeting endonuclease may be a zinc finger nuclease. Typically, a zinc finger nuclease comprises a DNA binding domain (i.e., zinc finger) and a cleavage domain (i.e., nuclease).

Such kits generally also include donor polynucleotides comprising the nucleic acids encoding the biosensor and any optional accessory sequences. The nucleic acids encoding the biosensor and any optional accessory sequences are flanked by upstream and downstream sequences that share substantial sequence identity with sequences at either side of the site of integration in the targeted chromosomal sequence. For more detail regarding ZFNs and methods of using to edit chromosomal regions see PCT/US2010/43167, the disclosure of which is incorporated by reference herein in its entirety.

In some embodiments, the kit may further comprise at least one additional component. Suitable components include transfection reagents, agents to enhance vector delivery, culture media for growing the cells, control vectors, dilution reagents, and the like. Typically, the kits also include instructions for transfecting the cells with the vectors comprising the nucleic acid encoding the biosensor and any optional accessory sequences.

### (IV) Cells Comprising the Biosensor

Yet another aspect of the disclosure encompasses cells comprising the disclosed biosensor. As detailed above in section (I), the biosensor is able to detect changes in the activity of a target signal transduction protein. Additionally, the cells disclosed herein express the biosensor at a level that is substantially similar to the level of the target signal transduction protein.

(a) Signal Transduction Proteins

The cells comprising the biosensor express the signal transduction protein of interest. Preferentially, the target signal transduction protein is an endogenous protein that is expressed from an endogenous chromosomal location. Alternatively, the target signal transduction protein may be an exogenously-introduced protein that is expressed from a chromosomally integrated nucleic acid sequence or an extrachromosomal nucleic acid sequence. The exogenous nucleic acid sequence may be overexpressed, however.

The identity of the target signal transduction protein can and will vary. Non-limiting examples of suitable signal transduction proteins include G protein-coupled receptors, transmembrane receptors, ligand-gated ion channels, voltage-gated ion channels, cytoplasmic protein kinases, serine-threonine kinases, protein phosphatases, phosphatidylinositol kinases, phospholipases and receptor tyrosine kinases, which may belong to the same or different groups for classification purposes. Classifying by compartmentation, there are intracellular receptors and cell-surface receptors.

In one embodiment, the target signal transduction protein may be an intracellular receptor. Intracellular receptors are soluble proteins localized within the nucleoplasm or the cytoplasm. Non-limiting examples include: cytoplasmic protein kinases, serine-threonine kinases, protein phosphatases, phosphatidylinositol kinases, phospholipases and non-receptor tyrosine kinases. Protein kinases transfer a phosphate group and covalently attach it to a serine, threonine, tyrosine, or histidine residue. At least 125 of the 500+human protein kinases are serine/threonine kinases. With most kinases acting on both serine and threonine, others act on tyrosine, yet some others act on all three. While the catalytic domain of these kinases is highly conserved, the sequence variation in the kinase encoding genes provides for specificity in recognition of distinct substrate. Phosphorylation of the substrate by protein kinase results in a functional change of the substrate, which may be changing enzyme activity, cellular location, or association with other proteins.

In another embodiment, the target signal transduction protein may be a cell-surface receptor with or without a transmembrane structure. Non-limiting examples of cell-surface receptors include G-protein coupled receptors, integrins, toll-like receptors, ligand-gated ion channel receptors, and receptor tyrosine kinases.

(i) G Protein-Coupled Receptors

Also known as GPCR, G protein-coupled receptors encompass a large group of transmembrane eukaryotic receptors that sense molecules outside the cell and activate inside signal transduction pathways. In one embodiment, the targeted signal transduction protein may be a GPCR. The G protein-coupled receptor is activated by a ligand or other signal mediator, which creates a conformational change in the receptor that causes activation of a G protein. The type of G protein activates more specific downstream effects. Non-limiting examples of the ligands include sensory signal mediators (e.g., light and olfactory stimulatory molecules); adenosine, bombesin, bradykinin, endothelin, γ-aminobutyric acid (GABA), hepatocyte growth factor (HGF), melanocortins, neuropeptide Y, opioid peptides, opsins, somatostatin, tachykinins, members of the vasoactive intestinal peptide family, and vasopressin; biogenic amines (e.g., dopamine, epinephrine, norepinephrine, histamine, glutamate (metabotropic effect), glucagon, acetylcholine (muscarinic effect), and serotonin); chemokines; lipid mediators of inflammation (e.g., prostaglandins, prostanoids, platelet-activating factor, and leukotrienes); and peptide hormones (e.g., calcitonin, C5a anaphylatoxin, follicle-stimulating hormone (FSH), gonadotropin-releasing hormone (GnRH), neurokinin, thyrotropin-releasing hormone (TRH), and oxytocin). GPCRs that act as receptors for stimuli that have not yet been identified are known as orphan receptors. G protein-coupled receptors are involved in many diseases, and are also the target of approximately 30% of modern therapeutic agents.

(ii) Integrins

During cell-cell or cell-ECM (extracellular matrix) signal transduction, an integrin receptor receives signals through its extracellular domain and induces a conformational change within itself and proteins at the cell surface to initiate signal transduction. Integrin-mediated signal transduction is achieved through a variety of intracellular protein kinases and adaptor molecules, which include integrin-linked kinase (ILK), Src-family kinases, and GTPases. In one embodiment, the targeted signal transduction protein may be an integrin.

(iii) Toll-Like Receptors

Toll-like receptors (TLR) are important components of the immune system in animals, plants and bacteria. All TLR members of have a common TIR (Toll-IL-1 receptor) domain. One subgroup of TLRs includes receptors for interleukins that are produced by macrophages, monocytes and dendritic cells and all have extracellular Immunoglobulin (Ig) domains. Another subgroup of TLRs bind directly or indirectly to molecules of microbial origin. A third subgroup of proteins containing TIR domains are exclusively cytosolic and mediate signaling from proteins of subgroups 1 and 2.

(iv) Liqand-Gated Ion Channel Receptors

Ligand-gated ion channel receptors (LGIC) are a group of transmembrane ion channels that are opened or closed in response to the binding of a chemical messenger, such as a neurotransmitter. For example, the nicotinic acetylcholine receptor is a LGIC activated through binding acetylcholine. Receptor's configuration alters upon ligand binding, which allowing Na⁺ ions to flow into the cell through the open channel until the cell membrane is depolarized and an action potential is initiated. In one embodiment, the targeted signal transduction protein is a LGIC.

(v) Receptor Tyrosine Kinases

In preferred embodiments, the signal transduction protein may be a receptor tyrosine kinase (RTK). RTKs are transmembrane receptors that, upon ligand binding, undergo dimerization and autophosphorylation of specific tyrosine residues. Adaptor proteins (e.g., those containing SH2 domains) recognize and bind the phosphorylated tyrosine residues, thereby activating downstream signaling processes.

RTKs have been classified into several families. One family is the epidermal growth factor receptor (EGFR) family (or ErbB protein family), which contains four structurally related receptor tyrosine kinases: ErbB-1 (or EGFR), ErbB-2, ErbB-3 and ErbB-4. Insufficient ErbB signaling in humans is associated with the development of neurodegenerative diseases, such as multiple sclerosis and Alzheimer's disease. In mice, loss of signaling by any member of the ErbB family results in embryonic lethality with defects in organs including the lungs, skin, heart, and brain. Excessive ErbB signaling is associated with the development of a wide variety of types of solid tumor. ErbB-1 and ErbB-2 are found in many human cancers and their excessive signaling may be critical factors in the development and malignancy of these tumors. In one embodiment, the target signal transduction protein may be selected from ErbB-1, ErbB-2, ErbB-3, ErbB-4 or their homologs in other organisms.

Another family of RTKs is the fibroblast growth factor receptor (FGFR) family. There are four fibroblast growth factor receptor (FGFR) genes in this family, which produce over 48 different isoforms of FGFR through alternate splicing during gene expression. In another embodiment, the target signal transduction protein may be selected from the FGFR isoforms or their homologs in other organisms.

RTKs also include members of vascular endothelial growth factor receptor (VEGFR) family. Members include VEGFR-1 (Flt-1), VEGFR-2 (KDR/Flk-1), and VEGFR-3. VEGFR-2 appears to mediate almost all of the known cellular responses to VEGF; VEGFR-1 modulates VEGFR-2 signaling; and VEGFR-3 mediates lymphangiogenesis in response to VEGF-C and VEGF-D. In still another embodiment, the target signal transduction protein may be selected from VEGFR-1, VEGFR-2, VEGFR-3, and variant thereof.

Another RTK family includes those activated by glial cell line-derived neurotrophic factor (GDNF), a family of extracellular signaling molecules or ligands. GDNF-activated RTKs include different isoforms of protein as the products of alternate splicing of the RET. RET loss of function mutations are associated with the development of Hirschsprung's disease, while gain of function mutations are associated with the development of various types of human cancer, including medullary thyroid carcinoma, multiple endocrine neoplasias type 2A and 2B. In an alternate embodiment, the target signal transduction protein may be a member of the RET receptor family or variant thereof.

Still another family of RTKs is the Eph receptor family, which comprises the largest sub-family of RTKs. There are at least 16 Known Eph receptors that can be activated by at least one of the 9 known ephrin ligands. Eph/ephrin families form a principle cell guidance system during vertebrate and invertebrate development, particularly in cell positioning and cell morphology. In one embodiment, the target signal transduction protein may be selected from the Eph receptor family.

(b) Types of Cells

The type of cell comprising the biosensor described above can and will vary. In general, the cell will be a eukaryotic cell. In some instances, the cell may be a primary cell, a cultured cell, or immortal cell line cell. Suitable cells include fungi or yeast, such as Pichia, Saccharomyces, or Schizosaccharomyces; insect cells, such as SF9 cells from Spodoptera frugiperda or S2 cells from Drosophila melanogaster; and animal cells, such as mouse, rat, hamster, non-human primate, or human cells. Exemplary cells are mammalian. The mammalian cells may be primary cells. In general, any primary cell that is sensitive to double strand breaks may be used. The cells may be of a variety of cell types, e.g., fibroblast, myoblast, T or B cell, macrophage, epithelial cell, and so forth.

When mammalian cell lines are used, the cell line may be any established cell line or a primary cell line that is not yet described. Suitable mammalian cell lines are presented above in section (III)(b).

In still other embodiments, the cell may be a stem cell. Suitable stem cells include without limit embryonic stem cells, ES-like stem cells, fetal stem cells, adult stem cells, pluripotent stem cells, induced pluripotent stem cells, multipotent stem cells, oligopotent stem cells, and unipotent stem cells.

In further embodiments, the cell may be a one-cell embryo. The embryo may be a vertebrate or an invertebrate. Suitable vertebrates include mammals, birds, reptiles, amphibians, and fish. Examples of suitable mammals include without limit rodents, companion animals, livestock, and non-primates. Non-limiting examples of rodents include mice, rats, hamsters, gerbils, and guinea pigs. Suitable companion animals include but are not limited to cats, dogs, rabbits, hedgehogs, and ferrets. Non-limiting examples of livestock include horses, goats, sheep, swine, cattle, llamas, and alpacas. Suitable non-primates include but are not limited to capuchin monkeys, chimpanzees, lemurs, macaques, marmosets, tamarins, spider monkeys, squirrel monkeys, and vervet monkeys. Non-limiting examples of birds include chickens, turkeys, ducks, and geese. Alternatively, the animal may be an invertebrate such as an insect, a nematode, and the like. Non-limiting examples of insects include Drosophila and mosquitoes.

(c) Biosensor and its Expression

The cells of the invention may comprise a variety of different biosensors, which are detailed above in section (I). The biosensor, however, is chosen such that it recognizes and binds a specific site or region in the target signal transduction protein. For example, the protein-binding domain of the biosensor may be a SH2 domain such that the biosensor may recognize and bind an activated RTK. Alternatively, the protein-binding domain of the biosensor may be a SH3 domain such that the biosensor may recognize and bind P13 kinase or phospholipase. Those of skill in the art will appreciate that numerous combinations are possible.

In some embodiments, the biosensor may be expressed from an extrachromosomal nucleic acid. That is, the nucleic acid encoding the biosensor and associated expression control sequences may be within an extrachromosomal vector (e.g., a plasmid vector), such that expression is regulated by the operably linked expression control sequence (i.e., promoter). Such cells may also be engineered to express a second fluorescent protein.

In preferred embodiments, the biosensor may be expressed from a chromosomally integrated nucleic acid. The nucleic acid encoding the biosensor and any optional associated sequences may be randomly inserted into a chromosome, such that expression is regulated by the operably linked promoter. Random integration of the biosensor may be accomplished through the use of viral vectors, such as, e.g., lentiviral particles. Alternatively, the nucleic acid encoding the biosensor may be inserted into a targeted location in a chromosome such that expression of the biosensor is regulated by an endogenous promoter. Targeted insertion of the biosensor may be accomplished by zinc finger nucleases and other targeting endonucleases.

Regardless of the mode of integration, the cells of the invention express the biosensor at substantially the same level as the signal transduction protein of interest. The phrase “at substantially the same level” as used herein means that the level of expression of the two proteins differs by less than about two-fold, or preferably less than about 50%. Stated another way, the cells of the invention do not overexpress the biosensor relative to the target signal transduction protein. Methods for measuring the level of protein expression are well known in the art. Cells that express the biosensor at substantially the same level as the target signal transduction protein are selected for and propagated using techniques well known in the art.

(d) Exemplary Cells

In preferred embodiments, the cell may be a mammalian cell comprising a biosensor that recognizes or “senses” a RTK. Generally, the signal transduction protein of interest is endogenous to the mammalian cell. In one embodiment, the cell comprises a biosensor comprising at least one SH2 domain. Preferentially, the SH2 domain is from a Grb2 protein. Thus, the SH2 domain recognizes and binds phosphotyrosine residues in activated EGFRs. In an exemplary embodiment, the cell comprises a biosensor comprising two SH2_{Grb2} domains. Moreover, this exemplary cell expresses the SH2 biosensor and EGFR at substantially similar levels. The fluorescent protein of the biosensor may be a green fluorescent protein, but other fluorescent proteins also may be used.

### (V) Methods for Detecting Activation of a Signal Transduction Protein

A further aspect of the disclosure provides methods for detecting activation of a signal transduction protein in real time. The method comprises providing a cell comprising a biosensor, as detailed above, contacting the cell with a signal, and monitoring the location of the biosensor in the cell. As detailed in the Examples, upon activation of a signal transduction protein, the protein-binding domain of the biosensor binds to the signal transduction protein, and, as a consequence, the cellular location of the biosensor changes. By monitoring the cellular translocation of fluorescent biosensor, one may monitor the activation and movement of the target signal transduction protein in live cells in real time.

(a) Providing a Cell Comprising the Biosensor

The first step of the method comprises providing a cell expressing the biosensor at substantially the same level as the target signal transduction protein. Suitable cells comprising the biosensor are detailed above in section (IV) and suitable biosensors are detailed above in section (I). In some embodiments, the cell may be an isolated cell and the method for detecting activation of a signal transduction protein may be carried out in vitro. In other embodiments, the cell may be within an organ, tissue, or organism and the method for detecting activation of a signal transduction protein may be carried out in situ or in vivo.

In general, the protein-binding domain of the biosensor recognizes and binds a specific site or region in the target signal transduction protein, wherein the binding changes upon activation or inactivation of the signal transduction protein. Accordingly, the cellular location of the biosensor changes spatially or temporally when the activity of the signal transduction protein changes. For example, the protein-binding domain of the biosensor may bind the target signal transduction protein when it is activated. Thus, upon activation of the signal transduction protein, the biosensor will translocate and bind to the signal transduction protein, thereby changing the cellular location of the biosensor. Alternatively, the protein-binding domain of the biosensor may bind the target signal transduction protein when it is an inactivate state. Thus, upon activation of the signal transduction protein, the biosensor will unbind the signal transduction protein and translocate to another location in the cell.

(b) Contacting the Cell with a Signal

The method further comprises contacting the cell comprising the biosensor with a signal that activates the signal transduction protein. Suitable signal transduction proteins are detailed above in section (IV)(a). The signal is chosen such that it activates the target signal transduction protein. Suitable signals include growth factors, hormones, neurotransmitters, small molecule ligands, cytokines, ions, and lipids. Exemplary signals include EGF, FGF, VEGF, GDNFs, TGFs, ephrins, BMPs, insulin, insulin-like growth factors, HGF, interleukins, epinephrine, norepinephrine, dopamine, serotonin, gastrin, cholecystokinin, thyroid hormone, erythropoietin, and so forth. In an exemplary embodiment, the signal may be EGF.

Depending on the nature of the signal used to activate the signal transduction protein, the cell is typically directly contacted with the signal. For example, the cell may be directly contacted with the signal by suspending the cells in a culture medium containing the signal. Alternatively, the cell may be directly contacted with the signal by administration of the signal to the organism comprising the cell. The administration may be via oral means or via parenteral means (e.g., injection).

The amount of signal contacted with the cell comprising the biosensor can and will vary. Those of skill in the art are familiar with means for determining the appropriate concentration of the signal to activate the target signal transduction protein.

The period of time the signal is contacted with the cell comprising the biosensor may vary. Typically, the signal transduction protein is immediately activated upon contact with the signal.

(c) Monitoring the Biosensor

The method further comprises monitoring the fluorescent biosensor in the cell, wherein a change in the cellular location of the biosensor indicates activation of the target signal transduction protein.

The monitoring step may be conducted in live cells or fixed cells. Preferentially, the monitoring is conducted in live cells in real time. Live-cell imaging techniques include a wide spectrum of imaging modalities, including fluorescence microscopy, widefield fluorescence, confocal, multiphoton, total internal reflection, FRET, lifetime imaging, superresolution, and transmitted light microscopy. In a preferred embodiment, the cell comprising the biosensor is imaged under low light fluorescence microscopy over time, such that the relocaliztion of the biosensor may be followed over the course of minutes or hours.

Upon activation of the target signal transduction protein, the binding of the protein-binding domain of the biosensor changes such that the location of the biosensor changes in the cell. For example, the biosensor may be uniformly distributed throughout the cytoplasm of the cell in the absence of the signal. Upon activation of the signal transduction protein, the biosensor may be translocated to the cell periphery, to the plasma membrane, to the cell nucleus, to endocytotic vesicles, to a cytoskeletal component, and the like. Alternatively, in the absence of the signal, the biosensor may be located at the plasma membrane but be relocated to other cellular compartments upon activation of the signal transduction protein.

In an exemplary embodiment, the signal transduction protein may be EGFR, the biosensor may comprise two SH2 protein-binding domains, and the signal may be EGF. Prior to contact with the signal, the SH2 biosensor may be uniformly distributed throughout the cytoplasm of the cell. Upon contact of the cell with EGF, the SH2 biosensor initially may translocate to the plasma membrane (where it binds the phosphorylated tyrosine residue in the EGFR) and then the biosensor may translocate to endocytotic vesicles (as the EGFR undergoes endocytosis). See FIG. 5 for a time course of the relocalization of the SH2 biosensor upon contact with EGF.

In some embodiments, the cell may further express a second fluorescent protein, as detailed above. The second fluorescent protein differs from the first fluorescent protein in the biosensor, such that the second fluorescent protein may be used an internal standard. For example, the ratio of the fluorescence signal of the fluorescent biosensor to the fluorescence signal of the second fluorescent protein may be used to account for differences in cell thickness and/or to improve the sensitivity of the fluorescence signal.

(VI) Methods for Screening Agents that May Modulate the Activity of a Signal Transduction Protein

Still another aspect of the disclosure encompasses methods for determining whether agents modulate the activity of signal transduction proteins. The method comprises providing a cell comprising a biosensor, contacting the cell with the agent, contacting the treated cell with a signal, and monitoring the biosensor in the cell relative to a cell not contacted with the agent, wherein a change in the location of the biosensor between the two cells indicates that the agent modulates the activity of the signal transduction protein. Examples 4 and 5 detail use of the method to identify agents that inhibit EGFR activity.

The method comprises providing a cell comprising the biosensor. Suitable cells comprising the biosensor are detailed above in section (V)(a).

The method further comprises contacting the cell comprising the biosensor with the agent. A variety of agents may be screened by the disclosed method. For example, agents that inhibit the activity of a signal transduction protein may be screened and identified. The types of agents screened will depend upon the identity of the signal transduction protein and the biosensor. For example, numerous small molecule compound libraries have been generated and are available commercially or from various sources.

The amount of agent contacted with the cell comprising the biosensor can and will vary. Those of skill in the art are familiar with means for determining the appropriate concentration of the agent.

The period of time the agent is contacted with the cell comprising the biosensor can and will vary. In general, the agent may be contacted with the cell comprising the biosensor for about 1 hour, about 2 hours, about 4 hours, about 8 hours, about 16 hours, or about 24 hours.

The method further comprises contacting the cells that were contacted with the agent with a signal, as detailed above in section (V)(b).

The last step of the method comprises monitoring the biosensor in the cell, in a manner similar to that detailed above in section (V)(c). The method for screening agents, however, differs from the previously described method in that the location of the biosensor in the agent-contacted cell is compared to the location of the biosensor in a control cell. The control cell is identical to the agent-contacted cell in all respects except it was not contacted with the agent. The agent may inhibit the activation of the signal transduction protein such that contact with the signal does change the localization of the biosensor. (See FIG. 7).

As various changes could be made in the above compounds, products and methods without departing from the scope of the invention, it is intended that all matter contained in the above description and in the examples given below, shall be interpreted as illustrative and not in a limiting sense.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

A “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

A “heterologous protein” is a protein that is not native (i.e., foreign) to the cell or organism of interest.

The terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The terms “polypeptide” and “protein” are used interchangeably to refer to a polymer of amino acid residues.

The term “recombination” refers to a process of exchange of genetic information between two polynucleotides. For the purposes of this disclosure, “homologous recombination” refers to the specialized form of such exchange that takes place, for example, during repair of double-strand breaks in cells. This process requires sequence similarity between the two polynucleotides, uses a “donor” or “exchange” molecule to template repair of a “target” molecule (i.e., the one that experienced the double-strand break), and is variously known as “non-crossover gene conversion” or “short tract gene conversion,” because it leads to the transfer of genetic information from the donor to the target. Without being bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized homologous recombination often results in an alteration of the sequence of the target molecule such that part or all of the sequence of the donor polynucleotide is incorporated into the target polynucleotide.

The term “sequence identity” refers to the extent in which two nucleotide sequences are invariant, i.e., the two sequences have the same nucleotide at the same position. Sequence identity is generally expressed as a percentage. Two nucleotide sequences that are identical in sequence and length have 100% sequence identity.

As used herein, the terms “target site” or “target sequence” refer to a nucleic acid sequence that defines a portion of a chromosomal sequence to be edited and to which a zinc finger nuclease is engineered to recognize and bind, provided sufficient conditions for binding exist.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

Alternatively, the degree of sequence similarity between polynucleotides can be determined by hybridization of polynucleotides under conditions that allow formation of stable duplexes between regions that share a degree of sequence identity, followed by digestion with single-stranded-specific nuclease(s), and size determination of the digested fragments. Two nucleic acid, or two polypeptide sequences are substantially similar to each other when the sequences exhibit at least about 70%-75%, preferably 80%-82%, more-preferably 85%-90%, even more preferably 92%, still more preferably 95%, and most preferably 98% sequence identity over a defined length of the molecules, as determined using the methods above. As used herein, substantially similar also refers to sequences showing complete identity to a specified DNA or polypeptide sequence. DNA sequences that are substantially similar can be identified in a Southern hybridization experiment under, for example, stringent conditions, as defined for that particular system. Defining appropriate hybridization conditions is within the skill of the art. See, e.g., Sambrook et al., supra; Nucleic Acid Hybridization: A Practical Approach, editors B. D. Hames and S. J. Higgins, (1985) Oxford; Washington, D.C.; IRL Press).

Selective hybridization of two nucleic acid fragments can be determined as follows. The degree of sequence identity between two nucleic acid molecules affects the efficiency and strength of hybridization events between such molecules. A partially identical nucleic acid sequence will at least partially inhibit the hybridization of a completely identical sequence to a target molecule. Inhibition of hybridization of the completely identical sequence can be assessed using hybridization assays that are well known in the art (e.g., Southern (DNA) blot, Northern (RNA) blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a reference nucleic acid sequence, and then by selection of appropriate conditions the probe and the reference sequence selectively hybridize, or bind, to each other to form a duplex molecule. A nucleic acid molecule that is capable of hybridizing selectively to a reference sequence under moderately stringent hybridization conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/reference sequence hybridization, where the probe and reference sequence have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press). Conditions for hybridization are well-known to those of skill in the art.

Hybridization stringency refers to the degree to which hybridization conditions disfavor the formation of hybrids containing mismatched nucleotides, with higher stringency correlated with a lower tolerance for mismatched hybrids. Factors that affect the stringency of hybridization are well-known to those of skill in the art and include, but are not limited to, temperature, pH, ionic strength, and concentration of organic solvents such as, for example, formamide and dimethylsulfoxide. As is known to those of skill in the art, hybridization stringency is increased by higher temperatures, lower ionic strength and lower solvent concentrations. With respect to stringency conditions for hybridization, it is well known in the art that numerous equivalent conditions can be employed to establish a particular stringency by varying, for example, the following factors: the length and nature of the sequences, base composition of the various sequences, concentrations of salts and other hybridization solution components, the presence or absence of blocking agents in the hybridization solutions (e.g., dextran sulfate, and polyethylene glycol), hybridization reaction temperature and time parameters, as well as, varying wash conditions. A particular set of hybridization conditions may be selected following standard methods in the art (see, for example, Sambrook, et al., Molecular Cloning: A Laboratory Manual, Second Edition, (1989) Cold Spring Harbor, N.Y.).

### EXAMPLES

The following examples illustrate certain aspects of the invention.

### Example 1

### EGFR Biosensors Comprising One or Two SH2 Domains

The generic structure of domain-based biosensors is illustrated in FIG. 1. The general structure of domain-based biosensors comprises two components: a protein-binding domain (or “binder”) that recognizes and binds a target protein and a fluorescent protein that permits monitoring of the cellular location of the biosensor. The protein-binding domain(s) may be located at the N-terminal end or the C-terminal end of the biosensor.

An EGFR biosensor comprising one SH2 domain, as shown in FIG. 2 upper panel, was constructed. The chosen protein-binding domain was the Scr homology 2 (SH2) of the Grb2 (growth factor binding 2) protein, which specifically binds EGF-activated RTKs. The fluorescent protein portion of the biosensor was either TurboGFP or TagGFP, each of which is an improved green fluorescent protein. A second biosensor comprising two SH2_{GRB2} domains was also constructed. See FIG. 2, lower panel. In this particular construct, the two SH2 domains were positioned side by side, with TurboGFP or TagGFP at the N-terminal.

A549 cells, which endogenously express EGFR, were transiently transfected with either the tagGFP-1×SH2 domain biosensor or the tagGFP-2×SH2 domain biosensor. Cells expressing the biosensors were exposed to 100 ng/mL of EGF and the location of the biosensors was monitored over time. As shown in FIG. 3, upper panel, cells expressing the 1×SH2 biosensor had much less pronounced relocalization of the biosensor (i.e., no granules were visible 45 min after exposure to EGF). In contrast, cells expressing low levels of the 2×SH2 biosensor displayed many fluorescent granules after 45 min (see FIG. 3, lower panel). The arrows point to cells expressing high levels of the 2×SH2 biosensor. Because of the high background of the unbound biosensor, relocalization of the biosensor was difficult to observe. In the right-most image in the lower panel of FIG. 3, fluorescent granules are slightly visible in the overexpressing cells (arrow). This suggests that it is important to match the expression levels of the biosensor and the target receptor for optimal signal sensitivity.

### Example 2

### Development of a Stable Cell Line

The nucleic acid encoding TagGFP-2×(SH2)_{Grb2} was cloned into a lentiviral vector. Lentiviral particles were prepared and used to stably transfect A549 cells with the TagGFP:2×(SH2)_{Grb2} biosensor. Single cell clones were selected and assayed for activity with treatment of EGF. A stable cell line homogenous for biosensor expression and EGF activity was selected. (see FIG. 4).

### Example 3

### TagGFP:2×(SH2)_{Grb2} Biosensor Relocalization Upon EGF Treatment

A549 cells stably transfected with the TagGFP:2×(SH2)_{Grb2} biosensor were treated with 100 ng/ml EGF and the spatial and temporal relocalization of the biosensor was monitored in living cells over time. The cell nuclei were labeled with 1 μM DRAQ5. As shown in FIG. 5, EGF treatment led to biosensor translocation to the plasma membrane (compare 0 min to 3 min) followed by increasing internalization into endocytotic vesicles (see discrete punctuate granules at 17 and 69 min).

The increased internalization of the biosensor over time was quantitated using granularity analysis in Metamorph. The number of granules was counted in individual cells. The cells were numbered as shown in FIG. 6, the first image in the top panel. The number in the left hand corner of each of the four images refers to the granule count for the selected cell: No. 4. Granule counts for each of the five cells over time are plotted in FIG. 6, bottom panel.

### Example 4

### Inhibitor of EGFR Blocked the TagGFP:2×(SH2)_{Grb2} Relocalization

To determine the specificity of the biosensor relocalization, cells expressing the SH2 biosensor were treated with tyrphostin AG 1478, which is a selective inhibitor or ErbB1, an EGFR family member. The cells were first treated with 1 μM AG 1478, and then treated with 1 μg/mL EGF. The control cells were only treated with 1 μg/mL EGF. The relocalization of the biosensor was monitored over time. As shown in FIG. 7, the translocation of TagGFP:2×(SH2)_{Grb2} to the plasma membrane was blocked in cells treated with AG 1478 in the images taken 3 min following EGF treatment. Further, the internalization of TagGFP:2×(SH2)_{Grb2} was also blocked in the cells treated with AG 1478, shown in the images taken at 50 min following EGF treatment. The results showed that the cells comprising the SH2 biosensor can be used to evaluate the inhibitors of signal transduction proteins.

### Example 5

### Identification of Other Activating Ligands

A variety of other ligands were tested for their ability to activate EGFR, as monitored by the SH2 biosensor. The tested ligands included: EGF, TGF-α, HGF, PDGF-AB, insulin, IGF-1, NGF-Beta, FGF-acidic, angiopoietin, MSP, Gas6, VEGF, and FLT-3. Most of the ligands were tested for activity with the TagGFP:2×(SH2)_{Grb2} biosensor in live cells. Heregulin-131 (EGF domain 176-246), however, was tested for activity with a TurboGFP:2×(SH2)_{Grb2} biosensor as compared to EGF, which was expressed as a second fluorescent protein. Table 1 presents the results. Activity (i.e., relocalization of the SH2 biosensor) was seen with EGF and TGF-α treatment, both of which are ligands specific for EGFR (ErbB-1). Heregulin-[3], a ligand specific for ErbB-3 and ErbB-4, did not show activity at 1 μg/mL (Table 1 and FIG. 8, top panel). HGF, a growth factor that binds HGFR, however, showed activity at 100 ng/mL, but much less than 100 mg/mL of EGF (Table 1 and FIG. 8, bottom panel).

TABLE 1

| Ligand | Activity |
|---|---|
| EGF | +++ |
| TGF-α | +++ |
| HGF | + |
| Heregulin-β1 | − |
| PDGF-AB | − |
| insulin | − |
| IGF-1 | − |
| NGF-Beta | − |
| FGF-acidic | − |
| angiopoietin | − |
| MSP | − |
| Gas6 | − |
| VEGF | − |
| FLT-3 | − |

### Example 6

### Ratiometric Imaging Improves Detection

A nucleic acid construct was generated in which a sequence encoding a RFP:2×(SH2)_{Grb2} biosensor linked to a sequence encoding GFP, wherein the two sequences were linked by sequence encoding a 2A peptide. See FIG. 9A. During translation, the 2A peptide region (˜20 amino acids) mediates “cleavage” at its own C-terminus, and produces two separate proteins: a RFP-2×(SH2)_{Grb2}-2A biosensor, and a separate GFP that serves as an internal marker or volume marker.

Cells expressing both proteins were exposed to 100 ng/mL of EGF. FIG. 9B presents images of the localization of the biosensor (red fluorescence) and the GFP volume marker (green fluorescence) over time, as well as the ratiometric image of the two (bottom panels). For ratiometric imaging, the fluorescence signal from the RFP biosensor was divided by the GFP signal in Metamorph. Ratiometric imaging improved detection of the biosensor because it removed the effect of changes in cell thickness on the fluorescence intensity of the biosensor and improved the sensitivity of detection at the thinner periphery of the cells.

### Example 7

### Targeted Integration into TUBA1B Locus Using ZFN Technology

The following example details a targeted insertion of a SH2 biosensor in the TUBA1B locus, such that expression of the biosensor was regulated by the endogenous tubulin promoter. For this, a pair of zinc finger nucleases (ZFNs) was designed to target a location in the TUBA1B coding region. One was designed to bind a sequence upstream and the second was designed to bind a sequence downstream of the target site. Upon binding, the ZFN pair will introduce a double-stranded break between the two ZFN binding sequences (see the top of FIG. 10). For more details regarding ZFNs and methods of using to edit chromosomal regions see PCT/US2010/43167, the disclosure of which is incorporated by reference herein in its entirely.

A donor plasmid was constructed that carried the sequence of the biosensor (i.e., GFP linked to two SH2_{Grb2} domains), which was linked at the 3′ end with a sequence encoding a 2A peptide. The sequence encoding the biosensor and 2A peptide (i.e., GFP-2×SH2_{Grb2}-2A) in the donor plasmid was flanked by upstream and downstream sequences of the ZFN cleavage site in the TUBA1B locus. The ZFNs and the donor plasmid were designed such that the SH2 biosensor coding sequence would be inserted in-frame with the endogenous tubulin sequence just downstream of the start codon. Upon activation of the tubulin promoter, one transcript comprising the tubulin start codon, the biosensor sequence, the 2A peptide sequence, and tubulin sequence would be made. During translation, however, two separate proteins would be made (i.e., the biosensor and tubulin), as depicted in the bottom of FIG. 10.

The donor plasmid and nucleic acids encoding the pair of ZFNs were transfected into A549 cells. The transfected cells were then cultured under standard conditions. Analysis of individual cell clones revealed uniform cytoplasmic GFP fluorescence, indicating the expression of the SH2 biosensor.

To examine activity of the SH2 biosensor, the cells were exposed to 100 ng/ml of EGF and imaged over time. FIG. 11 presents a time course of the translocation of the SH2 biosensor. Initially, the SH2 biosensor was translocated to the plasma membrane and then the SH2 biosensor was internalized by endocytosis.

### Example 8

### Targeted Integration into ACTB Locus Using ZFN Technology

To determine whether the level of expression of the biosensor affected its ability to track the relocalization of its target protein, a SH2 biosensor was inserted into a sequence under the control of a strong promoter. For this, the ACTB locus, which encodes β-actin, was chosen. ZFNs were designed to target the ACTB locus and introduce a cut site just downstream of the start codon. A donor plasmid was designed to provide the SH2_{Grb2} biosensor sequence, as well as tag the endogenously produced β-actin (i.e., GFP-2×SH2_{Grb2}-2A-RFP). The nucleic acids were introduced into A549 cells, and two fluorescent proteins were made (i.e., biosensor and RFP-actin).

Cells were exposed to 100 ng/ml of EGF and imaged. FIG. 12 presents a time course of the translocation of the GFP-biosensor and the location of RFP-actin. In many cells it was difficult to track the relocalization of the biosensor to the plasma membrane and endocytotic vesicles (see top panel in FIG. 12). In fact, GFP granules were visible only in cells with low levels of fluorescence (see arrow in FIG. 12). This example revealed that when high levels of the biosensor were produced, it was difficult to monitor the relocalization of the biosensor because of the high levels of unbound or “free” biosensor.

1. A cell comprising a biosensor for detecting a change in activity of an endogenous signal transduction protein, the biosensor comprising at least one protein-binding domain and a first fluorescent protein, and the cell being selected such that the biosensor is expressed at a level that is substantially similar to the level of the endogenous signal transduction protein.

2. The cell of claim 1, wherein the endogenous signal transduction protein is chosen from a receptor tyrosine kinase, a G protein-coupled receptor, a transmembrane receptor, a ligand-gated ion channel, a voltage-gated ion channel, a cytoplasmic protein kinase, a serine-threonine kinase, a protein phosphatase, a phosphatidylinositol kinase, and a phospholipase.

3. The cell of claim 2, wherein the receptor tyrosine kinase is chosen from an EGFR, a FGFR, a VEGFR, a RET receptor, and an Eph/ephrin receptor.

4. The cell of claim 1, wherein the protein-binding domain is chosen from SH2, SH3, 14-3-3, PDZ, PTB, WW, EVH, VHS, FHA, EH, FF, BRCT, Bromo, Chromo, GYF, C2, MH2, WD40, and variants thereof.

5. The cell of claim 1, wherein the first fluorescent protein is chosen from a green fluorescent protein, a blue fluorescent protein, a cyan fluorescent protein, a yellow fluorescent protein, an orange fluorescent protein, and a red fluorescent protein.

6. The cell of claim 1, wherein the biosensor is expressed from a chromosomally integrated nucleic acid or an extrachromosomal nucleic acid.

7. The cell of claim 6, wherein the chromosomally integrated nucleic acid was integrated randomly using a lentivirus.

8. The cell of claim 6, wherein the chromosomally integrated nucleic acid was integrated in a targeted location using a targeting endonuclease.

9. The cell of claim 1, wherein the cell also expresses a second fluorescent protein at the same level as the biosensor, the second fluorescent protein being different from the first fluorescent protein in the biosensor.

10. The cell of claim 1, wherein the cell is a human cell or a mammalian cell.

11. The cell of claim 1, wherein the endogenous signal transduction protein is EGFR and the protein-binding domain comprises two SH2 domains.

12. The cell of claim 11, wherein each SH2 domain is from a Grb2 protein.

13-38. (canceled)

39. A lentiviral particle, the lentiviral particle comprising a nucleic acid encoding a biosensor comprising at least one protein-binding domain and a first fluorescent protein.

40. The lentiviral particle of claim 39, wherein the protein-binding domain is chosen from SH2, SH3, 14-3-3, PDZ, PTB, WW, EVH, VHS, FHA, EH, FF, BRCT, Bromo, Chromo, GYF, C2, MH2, WD40, and variants thereof.

41. (canceled)

42. The lentiviral particle of claim 39, wherein the nucleic acid encoding the biosensor is operably linked to an expression control sequence chosen from a CMV promoter, a tetracycline-inducible promoter, a SV40 promoter, a PGK promoter, a MMTV promoter, a metallothionein-1 promoter, an adenovirus Ela promoter, an immediate early promoter, an immunoglobulin heavy chain promoter, and a RSV-LTR promoter.

43. (canceled)

44. The lentiviral particle of claim 39, wherein the nucleic acid encoding the biosensor is linked to a second nucleic acid encoding a second fluorescent protein by a sequence encoding a 2A peptide, wherein the second fluorescent protein differs from the first fluorescent protein.

45. (canceled)

46. The lentiviral particle of claim 39, wherein the protein-binding domain comprises two SH2 domains.

47. The lentiviral particle of claim 46, wherein each SH2 domain is from a Grb2 protein.

48. A kit for generating a cell comprising a biosensor, the kit comprising (a) a plurality of cells and (b) a plurality of lentiviral particles, each lentiviral particle comprising a nucleic acid encoding the biosensor, the biosensor comprising at least one protein-binding domain and a first fluorescent protein.

49. The kit of claim 48, wherein the biosensor is able to detect a change in activity of an endogenous signal transduction protein.

50-58. (canceled)
